(19) United States
(12) Patent Application Publication
(10) Pub. No.: US 2016/0136211 A1
(43) Pub. Date: May 19, 2016

(54) METHODS FOR THE TREATMENT OF BLADDER CANCER

(71) Applicant: VIRALYTICS LIMITED, Sydney, NSW (AU)

(72) Inventors: Darren Raymond Shafren, Merewether, NSW (AU); Gough AU, Islington, NSW (AU); Hardev Pandha, Ashtead, Surrey (GB); Guy Simpson, Epsom, Surrey (GB)

(21) Appl. No.: 14/896,913

(22) PCT Filed: Jun. 13, 2014
(86) PCT No.: PCT/AU2014/000611

§ 371 Date: Dec. 8, 2015

Related U.S. Application Data
(60) Provisional application No. 61/836,083, filed on Jun. 17, 2013.

(51) Int. Cl. A61K 35/768; A61K 31/7068; A61N 5/10; A61K 31/407

(52) U.S. Cl. CPC A61K 35/768; A61K 31/407; A61K 31/7068; A61N 5/10

(57) ABSTRACT

The present invention relates to methods of treating bladder cancer with human enterovirus C (HEC) in combination with chemotherapy or radiation therapy. The present invention also relates to methods for increasing susceptibility of a cancer cell to infection by HEC.

### FIELD

The present invention relates to methods of treating bladder cancer with human enterovirus C (HEC) in combination with chemotherapy or radiation therapy. The present invention also relates to methods for increasing susceptibility of a cancer cell to infection by HEC.

### INTRODUCTION

Bladder cancer (also referred to as urothelial carcinoma of the urinary bladder) is the fourth and ninth most common cancer amongst men and women, respectively, in Europe and North America, with an estimated global prevalence of 2.7 million. Bladder cancer results in significant mortality, with overall 5-year survival rates of only 57% and 47% for men and women, respectively, when presenting with muscle-invasive disease. The disease has two distinct identities. Most commonly it presents with superficial disease (stages Tis, Ta, T1) which may be relatively non-aggressive (papillary) and unlikely to cause morbidity. In contrast a proportion of patients present with high grade (non-papillary) disease characterized by a propensity to recur, invade and metastasize. Local progression (T2-4) disease requires bladder removal (cystectomy), radiotherapy or chemoradiotherapy but control rates are modest and morbidity is high. Disseminated disease (nodal or distant metastatic) may be palliated with chemotherapy but there is a lack of significantly effective treatment options.

Research into the biology and treatment of non-muscle invasive (NMIBC) or superficial bladder cancer has been minimal compared to many other malignancies. In addition to its impact on patients, the disease presents a significant economic burden on health systems with a mean estimated treatment and surveillance cost of $200,000 per patient from the time of diagnosis, making it the most expensive of all human cancers to treat from diagnosis to death. No treatment in the last decade has made significant improvements in patient survival; furthermore no predictive biomarkers can guide the physician which patients may have any benefit from systemic chemotherapy (in the neoadjuvant, adjuvant or palliative setting).

Following transurethral resection (TUR), live intravesical Bacille Calmette Guerin (BCG) has been the standard of care for maintenance treatment of superficial bladder cancer for decades. Studies have supported a schedule of monthly maintenance BCG instillations after an induction regime of six weekly instillations; chronic maintenance administration appears to be especially important. The use of BCG in this way is associated with reduced rates of recurrence and increase in progression free survival. Intravesical BCG regimens have evolved empirically rather than mechanistically, and a full understanding of the effect of BCG on tumour biology remains elusive. BCG is problematic in terms of its toxicities, which can be severe, and which include cystitis, prostatitis, granuloma formation, fever, pain, rigors and systemic BCG dissemination. There is a need for less- or non-toxic effective agents for the treatment of bladder cancer.

Intravesical chemotherapy has also been well studied. Whilst this is less toxic than intravesical BCG, it is definitively less effective. The most commonly used agents are mitomycin C (MMC) and gemcitabine, with other drugs at various stages of development. The available portfolio of biologic and cytotoxic options in NMIBC has been rationalised into risk-adapted clinical treatment guidelines. However there remains an absence of definitive evidence that current intravesical therapy is able to achieve permanent disease control, and a significant proportion of patients eventually require cystectomy, and/or succumb to invasive disease.

Coxsackievirus A21 (CVA21) has recently been shown to be an efficient oncolytic agent that specifically targets and rapidly lyzes human malignant melanoma, (Shafren et al. 2004; Au et al. 2005), myeloma (Au et al. 2007), prostate cancer (Berry et al. 2008) and breast cancer which express high levels of the CVA21 cellular uptake receptors both in vitro and in vivo. In addition, a Phase I clinical trial in late stage melanoma patients has recently been completed, and has demonstrated that intratumorally administered CVA21 is well tolerated in humans, and that 55.55% of patients experienced stabilization or reduction in injected tumour volumes, leading to a phase II trial in this setting. In a current Phase II clinical trial in late stage melanoma patients, intralesional CVA21 treatment has demonstrated activity in both injected lesions and non-injected distant lesions, while generally being well-tolerated.

There remains a need for new and improved methods for the treatment, alleviation, or prevention of bladder cancer and for methods of improving survival in subjects with bladder cancer.

### SUMMARY OF THE INVENTION

In one aspect the invention provides a method for the treatment of bladder cancer in a subject, the method comprising administering to said subject a therapeutically effective amount of a human enterovirus C (HEC) in combination with radiotherapy or chemotherapy.

In an embodiment the HEC recognises the cell adhesion molecule intercellular adhesion molecule-1 (ICAM-1) for infectivity of a cell.

In an embodiment the HEC a Coxsackievirus.

In an embodiment the human enterovirus C is selected from the group consisting of Coxsackievirus A13 (CVA13), Coxsackievirus A15 (CVA15), Coxsackievirus A18 (CVA18), and Coxsackievirus A21 (CVA21).

In an embodiment the human enterovirus C is Coxsackievirus A21 (CVA21).

In one aspect the invention provides a method for the treatment of bladder cancer in a subject, the method comprising administering to said subject a therapeutically effective amount of Coxsackievirus A21 (CVA21) in combination with radiotherapy.

In one aspect the invention provides a method for the treatment of bladder cancer in a subject, the method comprising administering to said subject a therapeutically effective amount of Coxsackievirus A21 (CVA21) in combination with chemotherapy. The chemotherapy comprises the administration to the subject of one or more chemotherapeutic agents.

In an embodiment the bladder cancer is non-muscle invasive bladder cancer.

In an embodiment the bladder cancer is characterised by one or more cells in which expression of ICAM-1 is elevated in comparison to non-cancer cells.

In an embodiment the bladder cancer is a resistant to a chemotherapeutic agent.

In an embodiment the bladder cancer is a cancer resistant mitomycin C.

The chemotherapeutic agent may be administered to the subject before the HEC is administered to the subject, concurrently with the HEC being administered to the subject, or after the HEC administered to the subject. In one embodiment the chemotherapeutic agent is administered to the subject before administration of the HEC virus.

In an embodiment the dose of chemotherapeutic agent administered to the subject is less than that considered to be an effective amount of the chemotherapeutic agent if administered as the sole treatment of the bladder cancer.

In an embodiment the dose of HEC administered to the subject is less than that considered to be an effective amount of the HEC if administered as the sole treatment of the bladder cancer.

The method may comprise multiple dosages of the HEC.

The method may comprise multiple dosages of the chemotherapeutic agent.

In an embodiment the method comprises administering a first dose of the chemotherapeutic agent to the subject, waiting a pre-determined time to permit up-regulated expression of ICAM-1, and optionally of DAF, in cells of the bladder cancer, then administering a first dose of the HEC to the subject.

In an embodiment the chemotherapeutic agent is administered to the subject between about one and eight hours before administration of the HEC.

In an embodiment the chemotherapeutic agent is administered to the subject between about two and six hours before administration of the HEC.

In an embodiment the chemotherapeutic agent is administered to the subject about four hours before administration of the HEC.

In an embodiment the chemotherapeutic agent is MMC.

In an embodiment the HEC is CVA21.

In an embodiment the method comprises administration of MMC to the subject by instillation for about one to about three hours, followed by administration of CVA21 within about 4 to 24 hours after completion of the MMC administration.

The radiation therapy may be administered to the subject before the HEC is administered to the subject, concurrently with the HEC being administered to the subject, or after the HEC administered to the subject.

In one embodiment the radiation therapy is administered to the subject before administration of the HEC.

In an embodiment the method comprises administering a first dose of radiation to the subject, waiting a pre-determined time to permit up-regulated expression of ICAM-1, and optionally of DAF, in cells of the bladder cancer, then administering a first dose of the HEC to the subject.

In one embodiment the radiation is administered to the subject about 12 to about 24 hours before administration of the HEC virus.

In one embodiment multiple doses of radiation are administered to the subject, such as two, three or four doses, before administration of the HEC virus.

In an embodiment the treatment provides increased survival time for a subject compared to estimated survival time in the absence of said treatment. In an embodiment the treatment provides retardation of tumour growth compared to estimated tumour growth in the absence of said treatment.

In an embodiment the subject is a human.

In one aspect the invention provides a method of increasing susceptibility of a cancer cell to infection with an HEC virus, the method exposing said cancer cell to a chemotherapeutic agent or to radiation before exposing said cell to the HEC virus.

In one aspect the invention provides a method for enhancing oncolytic treatment of a subject having bladder cancer, wherein the oncolytic treatment comprises administration of a HEC virus to said subject, the method comprising administering to said subject a chemotherapeutic agent prior to administering to said subject the HEC virus.

In one aspect the invention provides a method for increasing expression of ICAM-1 in a cancer cell, the method comprising exposing said cell to a chemotherapeutic agent.

In an embodiment the HEC virus is administered to said patient intravesically.

In an embodiment the chemotherapeutic agent is administered to said patient intravesically.

In one aspect the invention provides a human enterovirus C (HEC), for use in combination with chemotherapy or radiation therapy for the treatment of bladder cancer.

In one aspect the invention provides use of a human enterovirus C (HEC) for the manufacture of a medicament for treatment of bladder cancer in combination with chemotherapy or radiation therapy.

In an embodiment the method optionally includes a bladder rinse or washout prior to administration of the virus. In an embodiment the rinse or washout may comprise instillation of a mild detergent solution capable of disrupting the glycosaminoglycan (GAG) layer of the urothelium. In an embodiment the mild detergent solution comprises a non-ionic detergent. In an embodiment the mild detergent solution comprises DDM (n-dodecyl-β-D-maltoside).

### BRIEF DESCRIPTION OF DRAWINGS

FIG. 1: Surface expression of ICAM-1 (CD54) and DAF (CD55) in bladder cell line panel, T24, RT112, VMCUB-1, 5637, KU19-19 (referred to as RU19-19 in figures), TCCSUP-1.

Cell lines are detailed in Table 1.

FIG. 2 a): The effect of the combination of CVA21 and Mitomycin C on T24 cells.

FIG. 2 b): ED50 for CVA21 only on panel of bladder cancer cell line.

FIG. 2 c): The effect of the combination of CVA21 and chemotherapy on cell proliferation was assessed by calculating combination index (CI) values using CalcuSyn software (Biosoft).

FIG. 3: Combination index (CI) values for single fraction radiation and CVA21 in bladder cancer cell lines T24 and 5637. By Loewe criteria, additivity is denoted by a CI of 1, synergy by values less than 1.

FIG. 4: QPCR for ICAM-1/DAF expression. a) On 5637 & T24 cancer cell lines 24 hrs after irradiation (Gy 4-10). b) On 5637 cancer cell line exposed to Mitomycin C.

FIG. 5: FACS analysis of ICAM-1/DAF express in bladder cancer cell line pulse with Mitomycin C (X0.5 fold IC50 x1, X2) for 1, 3, 7 and 24 hrs.

FIG. 6: Synergy between CVA21 and chemotherapeutic agent MMC in bladder cancer cell line 5637. (b) Percent cell survival of 5637 cells over a range of multiplicities of infection (MOI) of CVA21 in combination with MMC over a range of concentrations from 0 μg/ml to 2.8 μg/ml. FIG. 6(d) Combination Index (CL) values for 5637 (FIG. 6d) cells exposed to combination CVA21 in combination with MMC over the indicated ranges. By Loewe criteria, additivity is denote by a CI of 1, synergy by values less than 1, and more than 1 is denoted antagonistic.

FIG. 7: Synergy between MMC and CVA21 on the bladder cell line T24. (a) Cell survival after MMC (0-3.36 ug/ml) and CVA21 (0-50 TCID₅₀/cell). (b) CI values across combination conditions showing synergy (CI<1) at low mitomycin concentrations, especially below 0.2 ug/ml.

FIG. 8: Enhanced viral replication of bladder cancer cells (cell line 5637) on exposure to MMC.

FIG. 9: Ex-vivo human bladder tumor tissue is highly infectable by CVA21. Tissue pieces originating from the same human bladder tumour were either infected with CVA21 or left uninfected. Immunofluorescence and immunostaining for coxsackievirus was performed 48 hours post infection. Viral infections are visualized by the bright red staining in A (the blue colour shows the DAPI stained nuclei of the cells) and by the brown 3,3′-Diaminobenzidine (DAB) staining in C. No positive viral staining was observed in the uninfected bladder tumor tissues (B and D).

FIG. 10: Patient derived bladder tumour cell line is highly infectable by CVA21. Coxsackievirus A21 is stable in human urine. Human cancer bladder tissue was disaggregated and primary tumour cells were isolated. These were tested for bladder tumour markers (Cytokeratin 7) (data not shown). Primary tumour cells were infected at varying MOIs and incubated at 37 C for 72 hours then photographed and analysed by MTS ([3-(4,5-dimethylthiazol-2-yl)-5-(3-carboxymethoxyphenyl)-2-(4-sulfophenyl)-2H-tetrazolium, inner salt) assay. (A) CVA21 MOI 3. (B) Uninfected cells. (C) MTS assay. (D) CVA21 (3×10⁶ TCID₅₀) was incubated at 37 C for one hour in healthy donor urine. Resulting virus was titrated by TCIID₅₀ on SK-MEL-28 cells for 5 days.

### ABBREVIATIONS

CI Combination Index
CVA21 Coxsackievirus A21
DAB 3,3′-Diaminobenzidine
DAF decay-accelerating factor
ICAM-1 intercellular adhesion molecule-1
MMC mitomycin C
MOI multiplicity of infection
MTS ([3-(4,5-dimethylthiazol-2-yl)-5-(3-carboxymethoxyphenyl)-2-(4-sulfophenyl)-2H-terrazolium, inner salt.
TCID₅₀ median tissue culture infectious dose, being the dose of virus that will produce cytopathic change in 50% of the host cells exposed to the virus.

### DESCRIPTION OF EMBODIMENTS

The invention will now be described in more detail, including, by way of illustration only, with respect to the examples which follow.

The following are some definitions that may be helpful in understanding the description of the present invention. These are intended as general definitions and should in no way limit the scope of the present invention to those terms alone, but are put forth for a better understanding of the following description.

In the context of this specification, the term “treatment” refers to any and all uses which remedy or alleviate a disease state or symptoms, prevent the establishment of disease, or otherwise prevent, hinder, retard, or reverse the progression of disease or other undesirable symptoms in any way whatsoever. For the avoidance of misunderstanding it is noted that “treatment” as used herein does not require complete cure or remission of the disease being treated.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers, but not the exclusion of any other step or element or integer or group of elements or integers. Thus, in the context of this specification, the term “comprising” means “including principally, but not necessarily solely”.

In the context of this specification, the term “about” when used in relation to a numerical value will be understood to convey the usual degree of variation known in the art for the measure being described. Where the art does not recognise a usual degree of variation for a measure or where it does and additional direction is nevertheless desirable, the term “about” as used herein will be understood to convey a variation of plus or minus 10% of the numerical value to which the term “about” is used.

In the context of this specification, the term “subject” or “patient” includes humans and individuals of any species of social, economic or research importance including but not limited to members of the genus ovine, bovine, equine, porcine, feline, canine, primates, rodents.

Any description of prior art documents herein, or statements herein derived from or based on those documents, is not an admission that the documents or derived statements are part of the common general knowledge of the relevant art in Australia or elsewhere.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

In the context of this specification, where a numerical range is provided it will be understood to encompass the stated end points of the range and all values between those end points, including any sub-ranges within those endpoints.

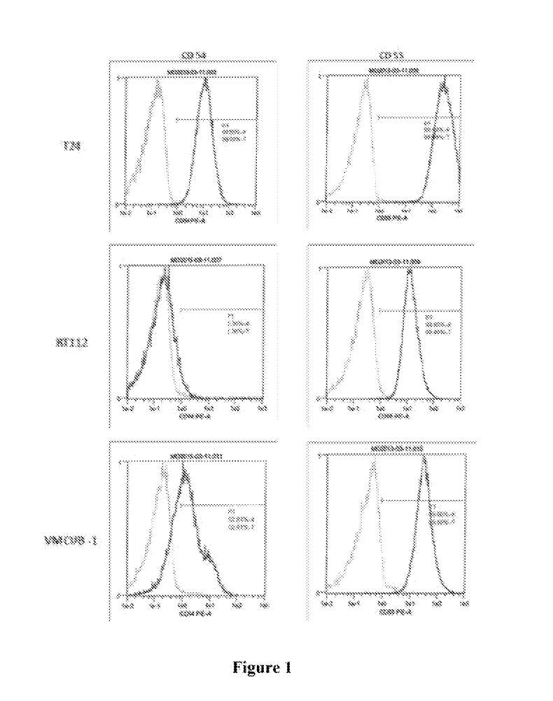

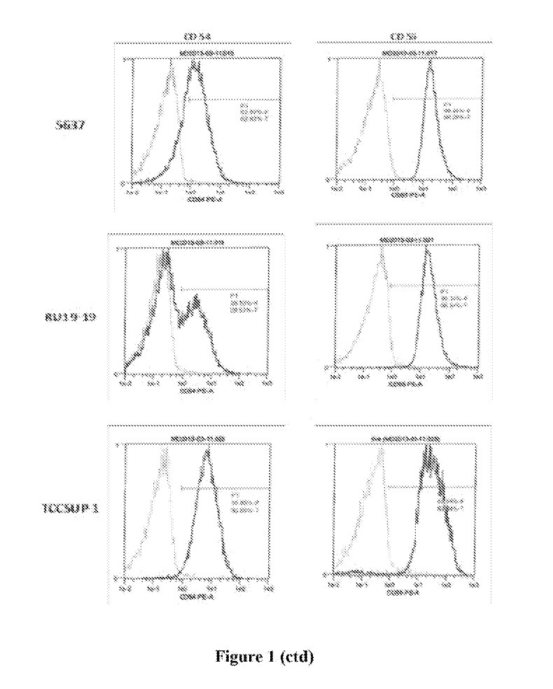

The inventors herein demonstrate application of coxsackievirus A21 (CVA21) for the treatment of bladder cancer, with particular reference to non-muscle invasive bladder cancer (NMIBC). In particular, the examples herein show most bladder cancer cell lines express ICAM-1 and DAF, and most are susceptible to CVA21 in vitro. The examples herein also show that upregulation of ICAM-1 can be achieved by adjunctive therapies. In particular, mitomycin C (MMC), an established intravesical agent, upregulates ICAM-1 expression and DAF expression at both the RNA and protein level. Furthermore, this translates into a synergistic therapy interaction between MMC and CVA21 (FIG. 1). Advantageously, these effects occur at very low concentrations of MMC, significantly below those subtended in urine and tissue by therapeutic intravesical MMC administration.

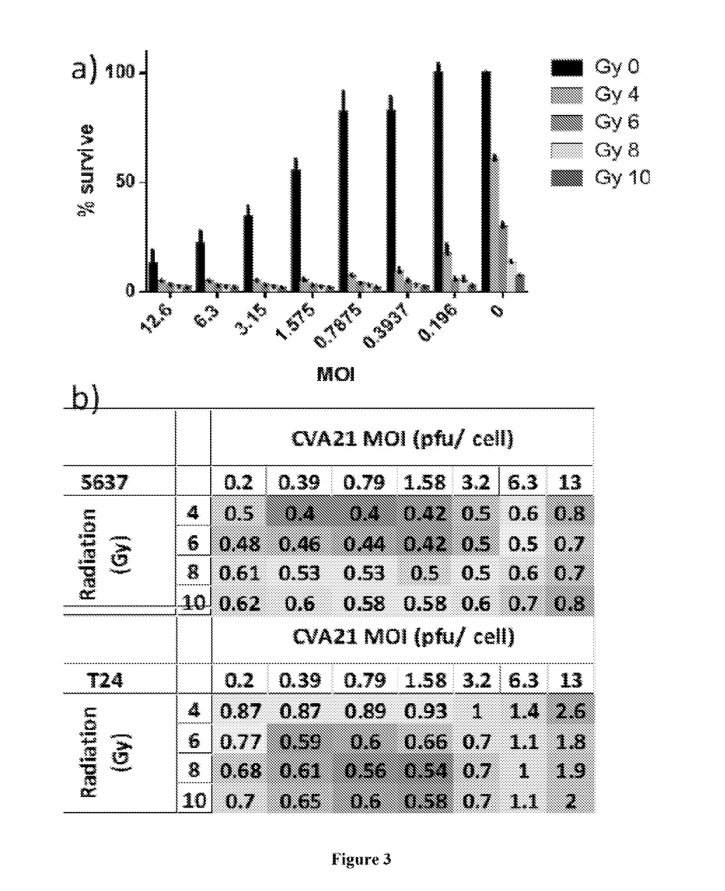

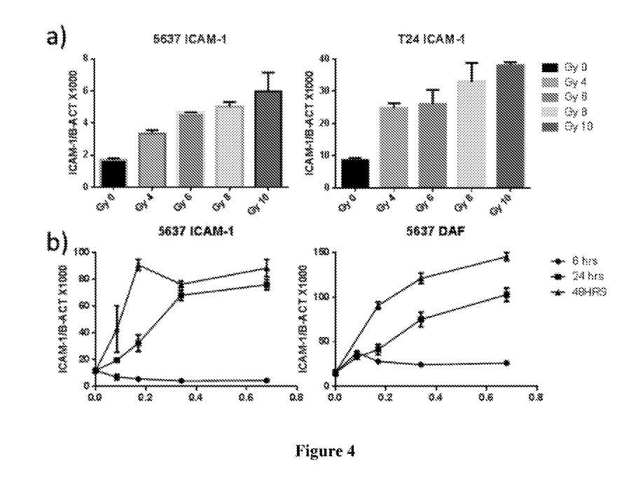

The inventors herein demonstrate application of coxsackievirus A21 (CVA21) for the treatment of bladder cancer, with particular reference to non-muscle invasive bladder cancer (NMIBC). The examples herein also show that up-regulation of ICAM-1 can be achieved by treatment of the cells with external radiation (4.0-8.0 Gy) (FIG. 4). Furthermore, this translates into a synergistic therapy interaction between radiation and CVA21 (FIG. 3).

CVA21 is a member of the human enterovirus C (HEC) family of viruses. Other notable members of the HEC family include the Coxsackieviruses, for example CVA13, CVA15, and CVA18. Each of CVA13, CVA15, CVA18 and CVA21 have been demonstrated to have oncolytic effect in the treatment of various solid cancers, such as breast cancer, prostate cancer, colorectal cancer and melanoma (Shafren et al, 2004; Au et al., 2005; Au et al., 2007; WO2001/037866 and entitled “A method of treating a malignancy in a subject and a pharmaceutical composition for use in same”; the contents of which is incorporated herein in its entirety by reference) and each interacts with the ICAM-1 receptor for infection of a host cell (Shafren et al, 1997) with decay accelerating factor (DAF) acting as a cooperative sequestration site (Shafren et al, 1997). Accordingly, the demonstration of a synergistic effect of CVA21 in combination with chemotherapeutic drugs, such as MMC or gemcitabine, or in combination with radiation therapy, will also apply to viruses functionally related to CVA21, such as CVA13, CVA15 and CVA18 and other human enterovirus C.

Any suitable source of the virus may be used in the methods of the invention. For example, various suitable strains of virus may be obtained from the American Type Culture Collection (ATCC), 10801 University Blvd., Manassas, Va. 20110-2209 USA, such as material deposited under the Budapest Treaty on the dates provided below, and is available according to the terms of the Budapest Treaty. Coxsackie group A virus, strain CVA13 ATCC No.: PTA-8854 Deposited 20 Dec. 10, 2007; Coxsackie group A virus, strain CVA15 (G9) ATCC No.: PTA-8616 Date of Deposit: Aug. 15, 2007; Coxsackie group A virus, strain CVA18 ATCC No.: PTA-8853 Deposited 20 Dec. 2007; Coxsackie group A virus, strain CVA21 (Kuykendall) ATCC No.: PTA-8852 Deposited 20 Dec. 2007.

Following infection, an oncolytic virus can kill a cancerous cell by direct lytic infection, induction of apoptosis or by initiating an immune response to viral antigens. An oncolytic virus is thus not limited to a single input dose and can undergo a multi-cycle infection, resulting in the production of large numbers of progeny virus. These progeny can spread either locally to adjacent tumour cells, or systemically to distant metastatic s sites. This feature of oncolytic therapy is particularly attractive for the treatment of inaccessible tumours or un-diagnosed micro-metastases. The demonstration herein that prior administration of a chemotherapeutic agent or prior radiation therapy enhances expression of ICAM-1 in the cancer cells, thereby rendering a cancer more susceptible to infection by a HEC, such as CVA21, thus offers, through such combination therapies, more potential for the use of oncolytic viruses for the treatment of bladder cancer. For example, cancer cells refractive to infection by the oncolytic virus may be rendered more susceptible to oncolysis.

The methods of the invention typically involve administration of a therapeutically effective amount of the virus and of the chemotherapeutic agent or radiation. The term “therapeutically effective amount” as used herein, includes within its meaning a non-toxic but sufficient amount of the virus, chemotherapeutic agent, or radiation, to provide the desired therapeutic effect. As noted herein, due to synergistic effects the amount of virus, chemotherapeutic agent, or radiation used may be less than that which would be used in a monotherapy (being a treatment of bladder cancer in a subject using just one of the virus, the chemotherapeutic agent or the radiation). The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

The method involves combination treatment of bladder cancer using a human enterovirus C in combination with a chemotherapeutic agent or radiation therapy. It will be understood that “in combination”, or similar terms, means that the virus and the chemotherapeutic agent or the virus and the radiation therapy are administered so as to have complementary therapeutic activities, and not necessarily that the virus and the chemotherapeutic agent or the virus and the radiation therapy are administered simultaneously to the subject. Typically, the chemotherapeutic agent will be administered to the subject prior to administration of the virus and the radiation therapy will be administered to the subject prior to administration of the virus. The virus and chemotherapeutic agent will typically therefore not be in physical combination prior to or when administered.

The virus is typically administered to the subject in the form of a pharmaceutical composition comprising virus and a pharmaceutically acceptable carrier. The composition may comprise the virus at any suitable concentration, such as in a concentration range of about 10⁵ viral particles per ml to about 10¹⁵ viral particles per ml, or about 10⁶ viral particles per ml, or about 10⁷ viral particles per ml or about 10⁸ viral particles per ml, or about 10⁹ viral particles per ml, or about 10¹⁰ viral particles per ml, or about 10¹¹ viral particles per ml, or about 10¹² viral particles per ml, about 10¹³ viral particles per mil, or about 10¹⁴ viral particles per ml, or about 10¹⁵ viral particles per ml.

A stock of the virus composition may be diluted to an appropriate volume suitable for dosing, for example to achieve the desired dose of viral particles administered in a desired volume. For example, a subject may be administered a dose of virus comprising about 10⁵ viral particles to about 10¹⁵ viral particles, or about 10⁶ viral particles, or about 10⁷ viral particles, or about 10⁸ viral particles, or about 10⁹ viral particles, or about 10¹⁰ viral particles, or about 10¹¹ viral particles, or about 10¹² viral particles, or about 10¹³ viral particles, or about 10¹⁴ viral particles, or about 10¹⁵ viral particles. The volume in which the virus is administered will be influenced by the manner of administration. For example, administration of the virus by injection would typically be in a smaller volume, for example about 0.5 ml to about 10 ml, compared to administration by intravesicular instillation, which may typically use about 10 ml to about 100 ml, for example about 20 ml, about 30 ml, about 40 ml, about 50 ml, about 60 ml, about 70 ml, about 80 ml or about 90 ml, or in volumes similar to known procedures for instillation of BCG for treatment of bladder cancer.

Compositions may additionally include a pharmaceutically acceptable diluent, excipient and/or adjuvant. The carriers, diluents, excipients and adjuvants must be “acceptable” in terms of being compatible with the other ingredients of the composition, and not unacceptably deleterious to the recipient subject.

The virus may be administered to the subject by any appropriate means, such as by injection. The injection may be systemically, parenterally, direct injection into the cancer, or intravesically. Typically, the administration of the virus is intravesically (infused directly into the bladder).

The virus may be administered as naked viral RNA encoding the virus, rather than viral particles, as described for example in PCT/AU2006/000051 entitled “Methods and composition for the treatment of neoplasms”, filed 17 Jan. 2006, published as WO2006/074526, the entire contents of which are incorporated herein by reference). In such an embodiment the viral RNA may be administered in the form of liposomes. Liposomes are generally derived from phospholipids or other lipid substances, and are formed by mono- or multi-lamellar hydrated liquid crystals that are dispersed in an aqueous medium. Any non-toxic, physiologically acceptable and metabolisable lipid capable of forming liposomes can be used. The compositions in liposome form may contain stabilisers, preservatives, excipients and the like. The preferred lipids are the phospholipids and the phosphatidyl cholines (lecithins), both natural and synthetic. Methods to form liposomes are known in the art, and in relation to this specific reference is made to: Prescott, Ed., Methods in Cell Biology, Volume XIV, Academic Press, New York, N.Y. (1976), p. 33 et seq., the contents of which is incorporated herein by reference.

The methods of the invention may optionally include a bladder rinse or washout prior to administration of the virus, for example to prepare the bladder for improved receptivity of the virus by removing or reducing the presence of agents which may reduce the efficacy of the virus. For example, the urothelium is protected by a glycosaminoglycan (GAG) layer, disruption of which may permit more efficient binding of the virus to cells and hence more efficient transduction of cells. In a non-limiting example DDM (n-dodecyl-β-D-maltoside), a nonionic mild detergent used as a food additive and solublizing agent, may be used to disrupt or remove the GAG layer at any appropriate concentration, for example at a concentration of about 0.1%, and thereby assist in facilitating transduction.

Chemotherapeutic agents for the treatment of bladder cancer are known. Typical agents include mitomycin C and gemcitabine. Mitomycin C causes delayed bone marrow toxicity and therefore it is usually administered at 6-weekly intervals. Prolonged use may result in permanent bone-marrow damage. It may also cause lung fibrosis and renal damage. In the methods of the instant invention, mitomycin C is used in combination therapy for bladder cancer with a human enterovirus C, such as CVA21. As shown in the examples herein, the effective dose of mitomycin C insuch combination therapy is reduced by comparison to that which is typically used in the treatment of bladder cancer. Hence, the instant invention may permit the use of mitomycin C in a manner in which typical deleterious side effects that have been observed in prior use of mitomycin C for treatment of bladder cancer are alleviated. This may permit, for example, a more aggressive use of mitomycin C than might otherwise have been available to the clinician when using mitomycin C at dosages typical of monotherapy.

The methods provided herein are for the treatment of bladder cancer. Typically the bladder cancer is non-muscle invasive bladder cancer (NMIBC) or transitional cell carcinoma (TCC, also urothelial cell carcinoma or UCC) which is a type of cancer that typically occurs in the urinary system: the kidney, urinary bladder, and accessory organs, and is the most common type of bladder cancer. The methods are also for the treatment of superficial bladder cancer.

The methods may comprise single or multiple doses of any one or more of the virus, the chemotherapeutic agent or the radiation therapy.

The methods of the invention may be used in combination with surgical treatment of the bladder cancer. For example bladder tumor resection may be followed by treatment of the subject using a combination method according to the invention. It is anticipated that this may prevent or reduce recurrence of the tumour.

The invention also relates to kits for use in the methods of the invention. In a basic form, the kit may comprise a pharmaceutical composition comprising the human enterovirus C and a pharmaceutically acceptable carrier, and instructions for the use of the composition, in combination with a chemotherapeutic agent or radiation, for the treatment of bladder cancer in a patient. The composition may be provided in any suitable container, such as for example a vial, ampoule or syringe. The composition may be provided lyophilised, freeze-dried, in liquid form or frozen state.

The kit may comprise any number of additional components. By way of non-limiting example, additional components may include (i) one or more anti-viral agents, such as Plecornil; (ii) one or more additional pharmaceutical compositions comprising an oncolytic virus; (iii) one or more additional therapeutic agents useful in the treatment of bladder cancer in a patient. The kit may additionally comprise a chemotherapeutic agent for use in the combination therapy, such as mitomycin C or gemcitabine. The kit may also comprise of the composition being contained in a single-use vial, a pre-loaded syringe for direct human administration, diluted in a physiological solution for intravenous infusion or in a concentrated form enabling suitable dilution with physiological solutions. Such solutions may be, for example, phosphate buffered saline or physiological concentrations of NaCl₂.

As used herein, the term “kit” refers to any delivery system for delivering materials. In the context of pharmaceutical compositions, such delivery systems include systems that allow for the storage, transport, or delivery of therapeutic agents (for example, oncolytic viruses in appropriate containers; or chemotherapeutic agents in appropriate containers) and/or supporting materials (for example, buffers, written instructions for use of the compositions, etc.) from one location to another. For example, kits include one or more enclosures, such as boxes, containing the relevant components and/or supporting materials.

The kit may be a fragmented kit. As used herein, the term “fragmented kit” refers to a delivery system comprising two or more separate containers that each contain a subportion of the total kit components. The containers may be delivered to the intended recipient together or separately. A fragmented kit may be suitable, for example, where one or more components, such as the virus or the chemotherapeutic agent, may optimally be stored and or transported under different conditions, such as at a different temperature, compared to one or more other components. Indeed, any delivery system comprising two or more separate containers that each contains a subportion of the total kit components are included in the term “fragmented kit.” In contrast, a “combined kit” refers to a delivery system containing all of the components of a reaction assay in a single container (e.g., in a single box housing each of the desired components). The term “kit” includes both fragmented and combined kits.

### EXAMPLES

The test article, Coxsackievirus A21 (CVA21) was provided by Viralytics Ltd. Research stocks for in vitro use were made from a vial of commercially prepared CVA21 in physiological saline.

Cell Lines.

Bladder cancer cell lines referred to in the Examples herein include T24, 5637, RT112, KU19-19, VMCUB-1, and TCCSUP-1. All cells were cultured at 37 C in a 5% CO₂ environment. Details of various cell lines are shown in Table 1. Cell lines marked with an asterisk were obtained from Professor Margaret Knowles (Cancer Research UK Clinical Centre, Leeds, UK).

TABLE 1

| | | | Histol- | | |
|---|---|---|---|---|---|
| | Species | | ogical | ECACC or | |
| Cell line | Source | Tissue | type | ATCC No | Media |
| EJ | Human | Bladder | TCC | 85061108 | DMEM |
| | | carcinoma | | | |
| T24 | Human | Bladder | TCC | 85061107, | McCoy's |
| | | carcinoma | | HTB-4 | |
| RT112 | Human | Bladder | TCC | 85061106 | MEME |
| | | carcinoma | | | |
| 5637 | Human | Bladder | TCC | *University | RPMI |
| | | carcinoma | | Leeds HTB-9 | |
| KU19-19 | Human | Bladder | TCC | *University | RPMI |
| | | carcinoma | | Leeds | |
| VMCUB-1 | Human | Bladder | TCC | *University | RPMI |
| | | carcinoma | | Leeds | |
| TCCSUP-1. | Human | Bladder | TCC | *University | MEME |
| | | carcinoma | | Leeds, HTB-5 | |

### Example 1

### Expression of ICAM-1 & DAF

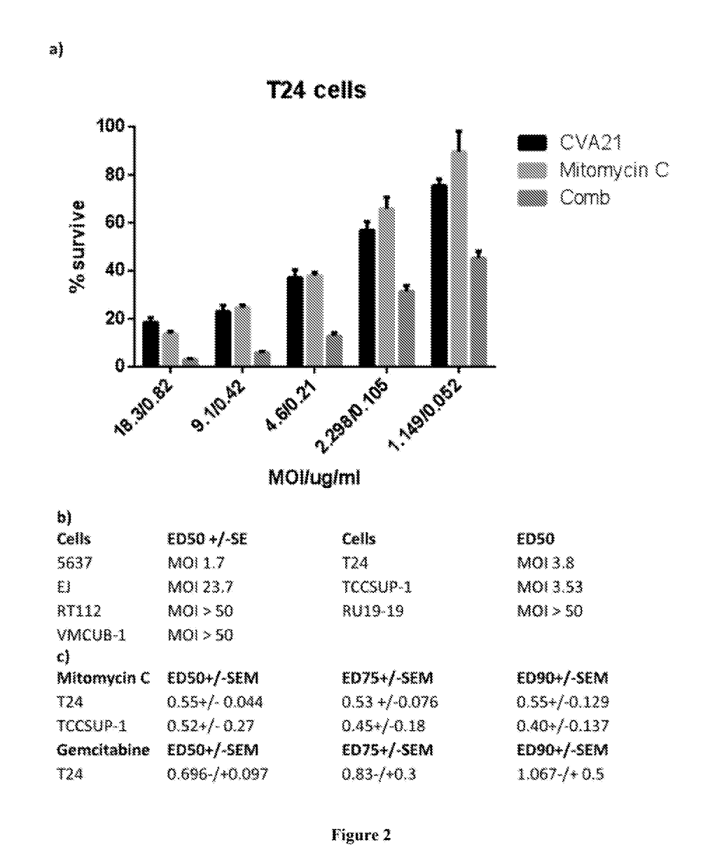

The cellular uptake of coxsackievirus A21 uptake is believed to be mediated by intercellular adhesion molecule 1 (ICAM-1, CD54) (Shafren et al. 1997), with decay accelerating factor (DAF, CD55) acting as a cooperative sequestration site (Shafren et al. 1997). This example investigated ICAM-1 expression in a bladder cancer cell line panel (FIG. 1). All bladder cell lines tested exhibit ICAM-1 expression except RT112 cells (FIG. 1). Notably the resistant cell lines KU19-19 and VMCUB-1 (FIG. 2b) also demonstrate ICAM-1 expression, suggesting that other phenotypic features of resistance may need to be explored for future patient stratification.

In brief, bladder cancer cells were plated at 5×10⁵ cells per well (2 ml) of a 6 well tray and incubated at 37° C. for 24 hrs. The cells were treated with Mitomycin C (2× fold IC50 1× fold IC50, 0.5× fold IC50) and each concentration incubated at 37° C. for 1, 3, 7 and 24 hrs. Therefore T24 cells were treated 0.75, 0.375, 0.1876 ug/ml Mitomycin C, 5637 cells were treated with 0.68, 0.34, 0.17 ug/ml Mitomycin C and KU19-19 cells were treated with 1.4876, 0.7438, 0.3719 ug/ml. The cells were trypsinised and centrifuged for 3 mins at 1500 rpm to a pellet and re-suspended in FACS Buffer (PBS containing 10% BSA and 1% sodium azide). 100 ul of cells were added to appropriate wells in a 96-well round-bottomed plate. Antibodies were prepared at 1:10 in FACS buffer CD54 PE (BD: 347977) mIgG2b, CD55 PE (BD: 555694) mIgG2a and Isotype controls. The plate was centrifuged for 2 mins at 2000 rpm and the supernatant flicked off. 40 ul of appropriate antibody or isotype control was added to wells. The plate was mixed on a plate shaker to ensure all cells were re-suspended and the cells incubated for 30 mins in dark at 4° C. Samples were read on a MACSQuant™ Analyzer (Bench top flow cytometer).

### Example 2

### Synergy Between CVA21 and Chemotherapy

CVA21 is an effective cytotoxic in three bladder cancer cell lines, T24, 5637 and TCCSUP-1 with typical ED50 values of 3.8, 1.7, and 3.52 TCID₅₀/cell respectively (FIG. 2b). Combining CVA21 with the chemotherapy agents Mitomycin C and Gemcitabine has shown surprising synergy. Using a fixed ratio design, the results demonstrate, from the 50% to the 90% effect levels, combination index values of 0.40-0.55 with Mitomycin C (FIG. 2c). Preliminary data using the same method has found from the 50% to the 75% effect levels, combination index values of 0.69-0.83 with Gemcitabine (FIG. 2b). In brief, 5637/T24/TCCSUP-1 cells were plated at 1×10⁴ cells per well (100 μL) of a 96 well tray and incubated at 37° C. for 24 hrs. Mitomycin C was diluted in 10% FCS medium in doubling dilutions from between 2.8 to 0.02 ug/ml for 5637 cells and between 3.36 to 0.03 ug/ml for T24 cells. CVA21 was then diluted between MOI 25-0.196 in doubling dilutions using each dilution of Mitomycin C. The cells were then treated with each dilution of CVA21/Mitomycin C and incubated for 72 hrs. The medium was removed and 100 μl of diluted MTS reagent (Promga) was added. The plates were then incubated for 1-4 hrs and absorbance read at 492 nm.

### Example 3

### Synergy Between CVA21 and Radiotherapy

Combining CVA21 with the radiotherapy has shown exceptional synergy. When 5637 cells were irradiated (4-10 Gy) then 24 hours later exposed to CVA21 (multiplicities of infection 0.961-12.6), clear synergy was seen (FIG. 3a). Dose matrix analysis showed that combination indices reached minima of approximately 0.4 (FIG. 3b). Synergy between radiation and CVA21 was confirmed in T24 cells (FIG. 3c). A comprehensive experimental and analytic method was implemented for this work which allows calculation of combination index values at all data points, and therefore identification of areas of high synergy across the whole response surface (Greco et al. 1995) (FIGS. 3b, 3c).

In brief, T24/5637 cells were plated at 0.25×10⁴/0.5×10⁴ cells per well (100 μL) of a 96 well tray and incubated at 37° C. for 24 hrs. Day 2—An extra 100 ul 10% FCS, media was added to the cells. Then they were treated with Rad (Gy 0, 4, 6, 8, 10) on a clinical Varian linear accelerator in St Luke's Cancer Centre, Royal Surrey Hospital UK. Day 2—The plates were returned to the lab and incubated at 37° C. for 24 hrs. Day 3—The medium was removed and 100 ul of CVA21 (MOI 12.5-0.1 in 2% FCS medium) was added and incubated at 37° C. for 72 hrs. Day 6—The medium was removed and 100 μl of fresh 10% FCS medium added and incubated for 24 hrs. Day 7—The medium was removed and 100 μl of diluted MTS reagent (Promega) was added. The plates were then incubated for 1-2 hr and read absorbance at 492 nm. For this work a comprehensive experimental and analytic method was implemented which allows calculation of combination index (CI) values at all data points, and therefore identification of areas of high synergy across the whole response surface (Greco et al. 1995).

### Example 4

### Up-Regulation of Expression of Viral Receptors ICAM-1 & DAF in Bladder Cancer Cell Lines after Exposure to Radiotherapy or Chemotherapy

Of significant interest, the results demonstrate that ICAM-1 expression is up-regulated by irradiation. A single fraction of 4 Gy increased ICAM-1 approximately two-fold in both T24 and 5637 cells (FIG. 4a). Further increases in doses resulted in incremental ICAM-1 transcriptional up-regulation.

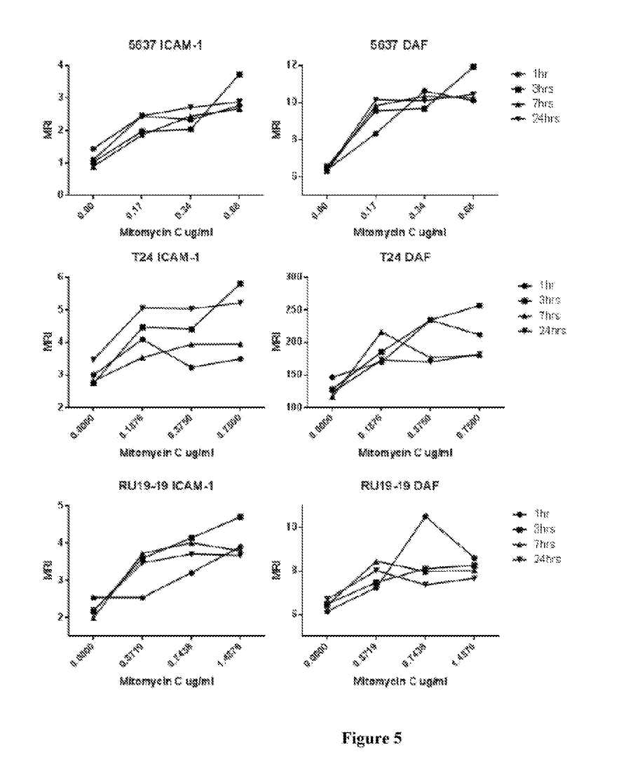

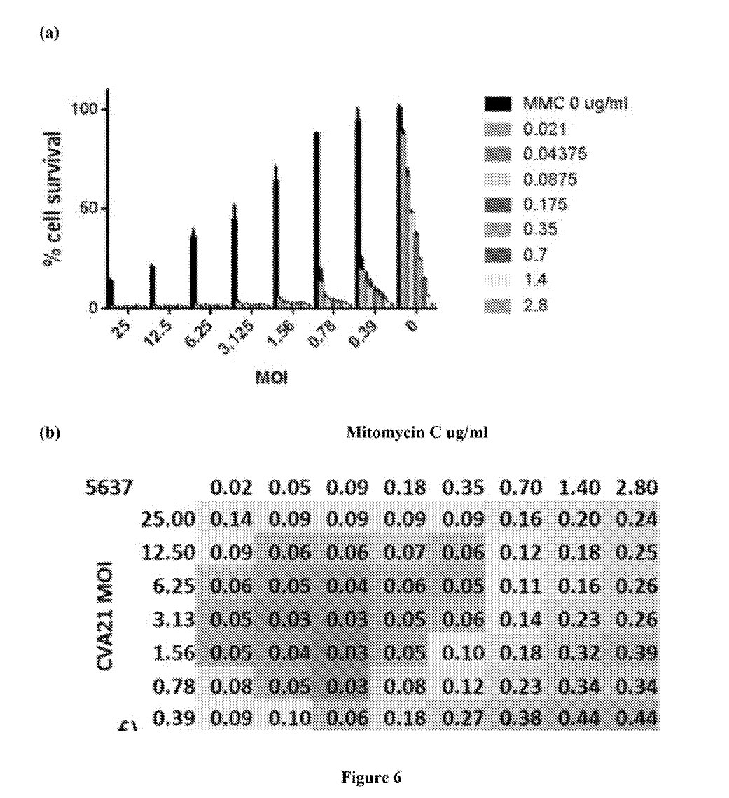

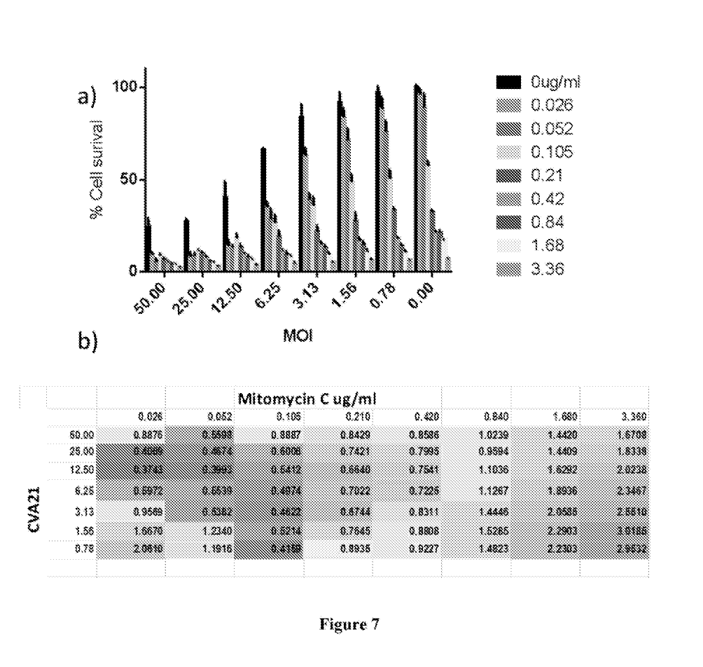

Exposure to the chemotherapy agent Mitomycin C, up-regulates both ICAM-1 and DAF at the RNA level (FIG. 4b). To mimic patient exposure to Mitomycin C T24, RU19-19 and 5637 cells were pulsed with drug for 1, 3, 7, 24 hrs and ICAM-1 and DAF expression was measured by FACS analysis at 24 hrs. The results demonstrate that ICAM-1 and DAF expression was strongly amplified after only a short pulse (1-3 hrs) of Mitomycin C on all three bladder cancer cell lines (FIG. 5).

This effect is reproducible, and holds for both concurrent and sequential dosing of MMC and CVA21. With a view to clinical translation, a variety of schedules for the potential combination of MMC and CVA21 have been explored by the inventors. The results indicate that a one hour pulse of MMC is sufficient for strong ICAM-1 amplification which is present from at least 4 hours after exposure, with modest incremental gains at later time points. Correspondingly synergy is well maintained (as compared with concomitant dosing) when CVA21 is administered 4 hours after MMC. This points towards a clinical schedule in which patients would receive an initial hour-long instillation of MMC followed by CVA21 instillation later the same day.

### Example 5

### Enhanced Viral Replication after Exposure to Mitomycin C

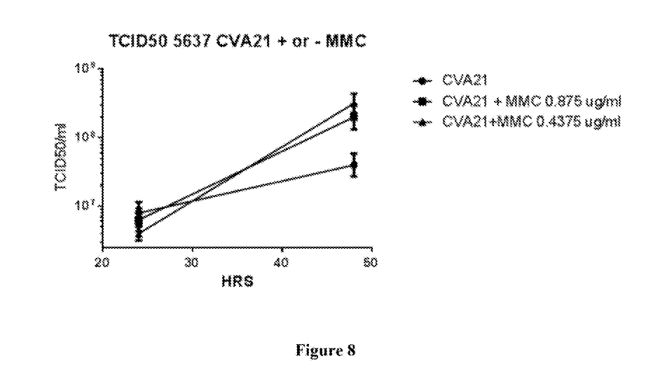

Exposure to MMC enhanced viral replication (FIG. 8). Monolayers of 5637 bladder cancer cells were plated and incubated at 37° C./5% CO₂ overnight. The media was removed, and CVA21 added at an MOI of 3 in 10% FCS medium containing 0, 0.4375 or 0.875 ug/ml Mitomycin C. The cells were then incubated at 37° C. for 24 or 48 hours. The plates were then frozen at −80° C. for 1 hour or o/n and then thawed after which cell CVA21 lysate was serially diluted 1:10 in 2% DMEM. The different concentrations of lysate were then added to SK-MEL-28 cells which had previously been plated at 1×10⁴ cells per well (100 μL) in a 96 well plate in 10% DMEM. The assay was then incubated at 37° C. for 5 days, after which the media was removed from the cells and 100 ul of 0.1% Glutaldehyde (Sigma) in PBS was added. After an incubation of 10 mins at RT, the Glutaldehyde solution was removed and 100 ul of 0.1% w/v Crystal Violet solution (in 20% Ethanol) was added in order to visualise the cells. Following another incubation of 10 mins at RT the excess Crystal Violet was removed with tap water. TCID50 was calculated by the Spearman & KArber algorithm as described in Hierholzer & Killington (1996), Virology Methods Manual, p. 374.

### Example 6

### Ex Vivo Human Bladder Tumour Tissue is Highly Permissive to Infection by CVA21

Primary bladder cancer tissue was received from the operating theatre of the Royal Surrey County Hospital UK in a dry pot. The tissue was cut into small pieces of between 2-4 mm and placed in 0.5 ml 10% FCS/DMEM with Pen/Strep and GLUT containing 3.875×10⁶ TCID₅₀ of CVA21. The infected tissue was incubated at 37° C., 5% CO₂ for 48 hrs. Tissue was then fixed in 10% neutral buffered formalin for 18-24 hours.

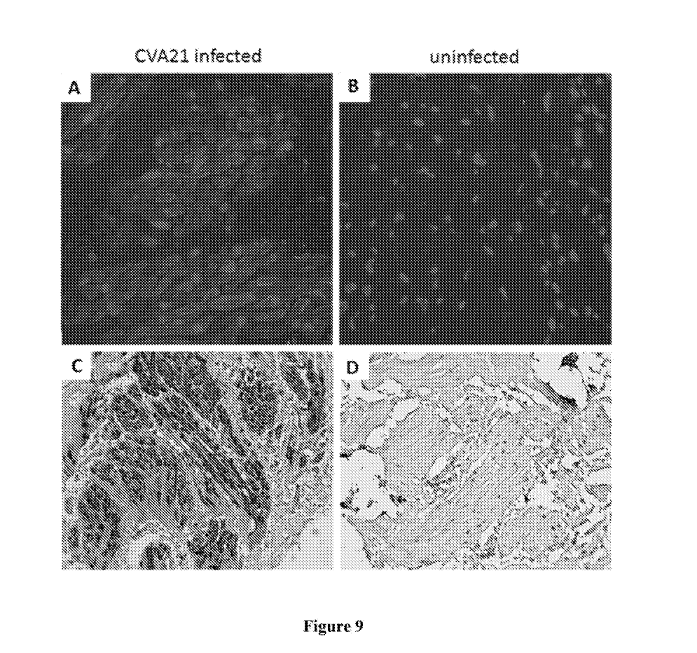

Tissue pieces originating from the same human bladder tumour were either infected with CVA21 or left uninfected. Immunofluorescence and immunostaining for coxsackievirus was performed 48 hours post infection. In FIG. 9, viral infections are visualized by the bright red staining in A (the blue colour shows the DAPI stained nuclei of the cells) and by the brown 3,3′-Diaminobenzidine (DAB) staining in C. No positive viral staining was observed in the uninfected bladder tumor tissues (FIGS. 9B and D).

In brief, bladder cancer tissue was fixed using 10% neutral buffered formalin for 18-24 hours. After fixation, the tissue block was embedded in paraffin, and 4 μm sections cut and affixed onto slides. The sections were dried overnight at 37° C., deparaffinized, and rehydrated. Endogenous peroxidase was blocked using methanol/0.3% H₂O₂ for 20 min. The sections were then subjected to heat mediated antigen retrieval in a microwave using citrate buffer (10 mM, pH 6.0). Following washing, the slides were blocked with 2.5% horse serum and endogenous biotin blocked using an AvidiniBiotin blocking kit (SP-2001, VectorLabs) according to the manufacturer's instructions. The primary antibody, anti-Enterovirus Ab (clone 5-D8/1; DAKO) was added at 1:10 and incubated overnight in a moist chamber. Slides were washed 3 times in PBS and positive staining visualised using the R.T.U. Vectastain Universal Elite ABC kit (VectorLabs) and DAB detection. Slides were then counterstained with haematoxylin before dehydrating in a series of alcohols and mounting with VectaMount (VectorLabs).

### Example 7

### Infection of Patient Derived Bladder Tumor with CVA21

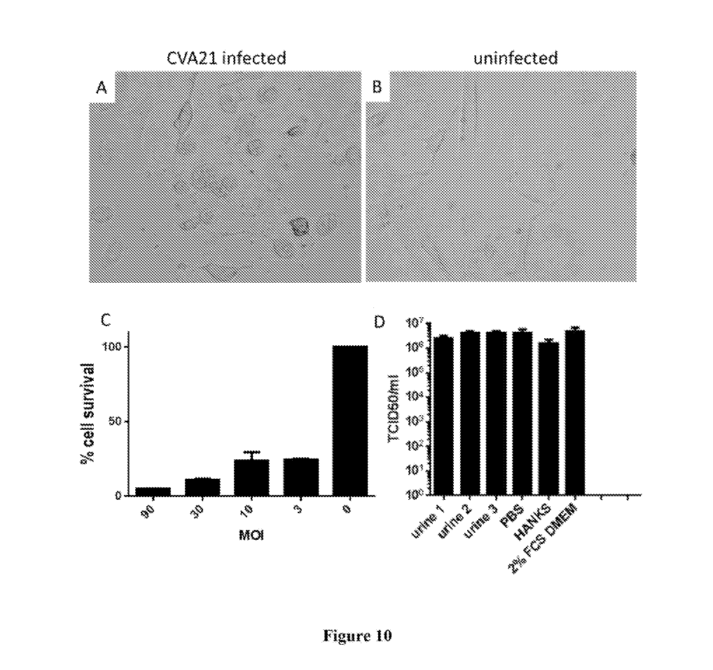

Human cancer bladder tissue was disaggregated and primary tumour cells were isolated. These were tested for bladder tumour markers (Cytokeratin 7) (data not shown). Primary tumour cells were infected at varying MOIs and incubated at 37 C for 72 hours then photographed and analysed by MTS ([3-(4,5-dimethylthiazol-2-yl)-5-(3-carboxymethoxyphenyl)-2-(4-sulfophenyl)-2H-tetrazolium, inner salt) assay. Results are shown in FIG. 10. (A) CVA21 MOI 3. (B) Uninfected cells. (C) MTS assay. (D) CVA21 (3×10⁶ TCID₅₀) was incubated at 37 C for one hour in healthy donor urine. Resulting virus was titrated by TCID₅₀ on SK-MEL-28 cells for 5 days.

In brief, SK-MEL-28 cells were plated at 1×10⁴ cells per well (100 μL) of a 96 well tray in 10% DMEM and incubate at 37° C. o/n. 37.5 ul of stock CVA21 virus (7.75e7 TCID₅₀/ml) was added 462.5 ul of normal health urine or Hanks or PBS or HANKS for 1 hrs at 37° C. After which urine/CVA21 was serially diluted 1:10 in 2% DMEM. The media was removed from the cells and 100 ul of each dilution was added to one of ten wells. The assay was then incubated at 37° C. for 5 days, after which the media was removed from the cells and 100 ul of 0.1% Glutaldehyde (Sigma) in PBS was added. After an incubation of 10 mins at RT, the Glutaldehyde solution was removed and 100 ul of 0.1% w/v Crystal Violet solution (in 20% Ethanol) was added in order to visualise the cells. After another incubation of 10 mins at RT the excess Crystal Violet was removed with tap water. TCID50 is calculated by the Spearman & Kärber algorithm. TCID50 is calculated by the Spearman & Kirber algorithm as described in Hierholzer & Killington (1996), Virology Methods Manual, p. 374.

### DISCUSSION

Combining CVA21 with either radiotherapy or chemotherapy synergistically enhances cytotoxicity in bladder cancer cell lines. Radiation and chemotherapy enhanced CVA21 viral replication and oncolysis, likely by increased expression of viral receptors ICAM-1 and DAF. Ex vivo human bladder tumour material and primary derived cell lines are highly infectable by CVA21. These results offer strong support for the efficacy of CVA21 plus chemotherapy or radiotherapy for the treatment of bladder cancer.

As demonstrated herein synergy is seen to occur between MMC and CVA21 at very low doses of CVA21, the MMC augmenting the therapeutic efficacy of the CVA21. Furthermore, the dose-sparing benefits of therapeutic synergy between the MMC and CVA21 and between the radiation and CVA21 reduce the toxicity risk from the partner agent and thereby expand the therapeutic index for patients.

### REFERENCES

Au, (2005). Int J Oncol 26(6): 1471-1476.
Greco (1995). Pharmacol Rev 47(2): 331-385.
Kirkali (2005). Urology 66(6 Suppl 1): 4-34.
Shafren (2004). Clinical cancer research 10(1 Pt 1): 53-60.
Shafren (1997). Journal of virology 71(1): 785-789.
Shafren (1997). “Coxsackievirus Journal of virology 71(6): 4736-4743.
Shelley (2004). BJU international 93(4): 485-490.
Sylvester (2006). European urology 49(3): 466-465; discussion 475-467.

What is claimed is:

1. A method for the treatment of bladder cancer in a subject, the method comprising administering to said subject a therapeutically effective amount of a human enterovirus C (HEC) in combination with radiotherapy or chemotherapy.

2. The method according to claim 1, wherein the HEC recognises the cell adhesion molecule intercellular adhesion molecule-1 (ICAM-1) for infectivity of a cell.

3. The method according to claim 1, wherein the HEC a Coxsackievirus.

4. The method according to claim 1, wherein the human enterovirus C is selected from the group consisting of Coxsackievirus A13 (CVA13), Coxsackievirus A15 (CVA15), Coxsackievirus A18 (CVA18), and Coxsackievirus A21 (CVA21).

5. The method according to claim 1, wherein the human enterovirus C is Coxsackievirus A21 (CVA21).

6. A method for the treatment of bladder cancer in a subject, the method comprising administering to said subject a therapeutically effective amount of Coxsackievirus A21 (CVA21) in combination with radiotherapy.

7. A method for the treatment of bladder cancer in a subject, the method comprising administering to said subject a therapeutically effective amount of Coxsackievirus A21 (CVA21) in combination with chemotherapy.

8. The method according to any one of claims 1 to 7, wherein the bladder cancer is non-muscle invasive bladder cancer (NMIBC).

9. The method according to any one of claims 1 to 8 wherein the bladder cancer is characterised by one or more cells in which expression of ICAM-1 is elevated in comparison to non-cancer cells.

10. The method according to any one of claims 1 to 9, wherein the bladder cancer is a cancer resistant to infection by said HEC in HEC monotherapy.

11. The method according to any one of claims 1 to 10, wherein the bladder cancer is a cancer resistant to infection by CVA21 in CVA21 monotherapy.

12. The method according to any one of claims 1 to 11, wherein the dose of HEC administered to the subject is less than that considered to be an effective amount of the HEC if administered as the sole treatment of the bladder cancer.

13. The method according to any one of claims 1 to 14, wherein said method comprises multiple dosages of the HEC.

14. The method according to claim 1 or 7, wherein chemotherapy comprises the administration to the subject of one or more chemotherapeutic agents.

15. The method according to claim 1 or 7, wherein the bladder cancer is a cancer resistant to a chemotherapeutic agent.

16. The method according to claim 1 or 7, wherein the bladder cancer is a cancer resistant mitomycin C (MMC) or gemcitabine.

17. The method according to claim 1 or 7, wherein the chemotherapeutic agent is administered to the subject before administration of the virus.

18. The method according to claim 1 or 7, wherein the dose of chemotherapeutic agent administered to the subject is less than that considered to be an effective amount of the chemotherapeutic agent if administered as the sole treatment of the bladder cancer.

19. The method according to claim 1 or 7, wherein said method comprises comprise multiple dosages of the chemotherapeutic agent.

20. The method according to claim 1 or 7, wherein the method comprises administering a first dose of the chemotherapeutic agent to the subject, waiting a pre-determined time to permit up-regulated expression of ICAM-1, and optionally of DAF, in cells of the bladder cancer, then administering a first dose of the HEC to the subject.

21. The method according to claim 1 or 7, wherein the chemotherapeutic agent is administered to the subject between about one and eight hours before administration of the HEC.

22. The method according to claim 1 or 7, wherein the chemotherapeutic agent is administered to the subject between about two and six hours before administration of the HEC.

23. The method according to claim 1 or 7, wherein the chemotherapeutic agent is administered to the subject about four hours before administration of the HEC.

24. The method according to claim 1 or 7, wherein the chemotherapeutic agent is MMC.

25. The method according to claim 1 or 7, wherein the chemotherapeutic agent is gemcitabine.

26. The method according to any one of claims 1 to 25, wherein the HEC is CVA21.

27. The method according to claim 1 or 7, wherein the method comprises administration of MMC to the subject by instillation for about one to about three hours, followed by administration of CVA21 within about 4 to 24 hours after completion of the MMC administration.

28. The method according to claim 1 or 6, wherein the radiation therapy is administered to the subject before administration of the virus.

29. The method according to claim 1 or 6, wherein the method comprises administering a first dose of radiation to the subject, waiting a pre-determined time to permit up-regulated expression of ICAM-1, and optionally of DAF, in one or more cells of the bladder cancer, then administering a first dose of virus to the subject.

30. The method according to claim 1 or 6, wherein the radiation is administered to the subject about 12 to about 24 hours before administration of the HEC virus.

31. The method according to claim 1 or 6, wherein multiple doses of radiation are administered to the subject, such as two, three or four doses, before administration of the virus.

32. A method of increasing susceptibility of a cancer cell to infection with an HEC virus, the method comprising exposing said cancer cell to a chemotherapeutic agent or to radiation before exposing said cell to the HEC virus.

33. A method for enhancing oncolytic treatment of a subject having bladder cancer, wherein the oncolytic treatment comprises administration of a HEC virus to said subject, the method comprising administering to said subject a chemotherapeutic agent prior to administering to said subject the HEC virus.

34. A method for enhancing oncolytic treatment of a subject having bladder cancer, wherein the oncolytic treatment comprises administration of a HEC virus to said subject, the method comprising administering to said subject one or more doses of radiation therapy prior to administering to said subject the HEC virus.

35. A method for increasing expression of ICAM-1 in a cancer cell, the method comprising exposing said cell to a chemotherapeutic agent.

36. A method for increasing expression of ICAM-1 in a cancer cell, the method comprising exposing said cell to one or more doses of radiation therapy.

37. The method according to any one of claims 1 to 34, wherein the virus is administered to said patient intravesically.

38. The method according to any one of claims 1 to 35, wherein the chemotherapeutic agent is administered to said patient intravesically.

39. The method according to any one of claims 1 to 38, wherein the method optionally includes a bladder rinse or washout prior to administration of the virus.

40. The method according to claim 39, wherein the rinse or washout comprises instillation of a mild detergent solution capable of disrupting the glycosaminoglycan (GAG) layer of the urothelium, optionally where the mild detergent solution comprises DDM (n-dodecyl-β-D-maltoside).
